(19) United States
(12) Plant Patent Application Publication
(10) Pub. No.: US 2004/0237163 P1
(43) Pub. Date: Nov. 25, 2004

(54) Coleus plant named 'Painted Pink'

(75) Inventor: Mitchell Donahue, Indian Trail, NC (US)

Correspondence Address: NORTH CAROLINA FARMS INC, 4205 FOREST DR, INDIAN TRAIL, NC, 28074, US

(73) Assignee: North Carolina Farms INC.

(21) Appl. No.: 10/445,211

(22) Filed: May 23, 2003

(51) Int. Cl. A01H 5/00

(52) U.S. Cl. PLT/373

(57) ABSTRACT

A distinct cultivar of Coleus plant named ‘Painted Pink’, characterized by its hot pink and maroon bi-colored leaves with maroon in the center surrounded by a wide hot pink margin; lack of flower development; tolerant to full sun conditions.

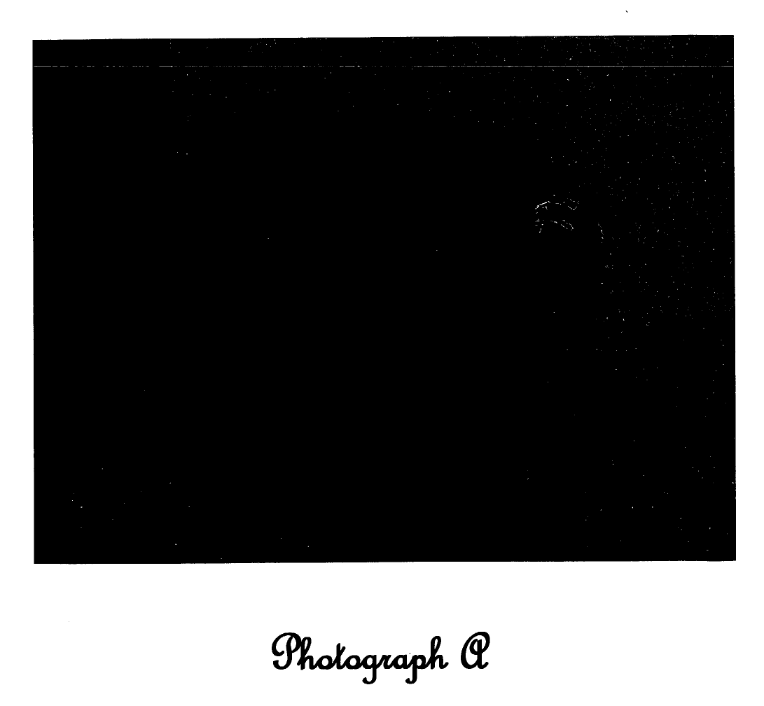

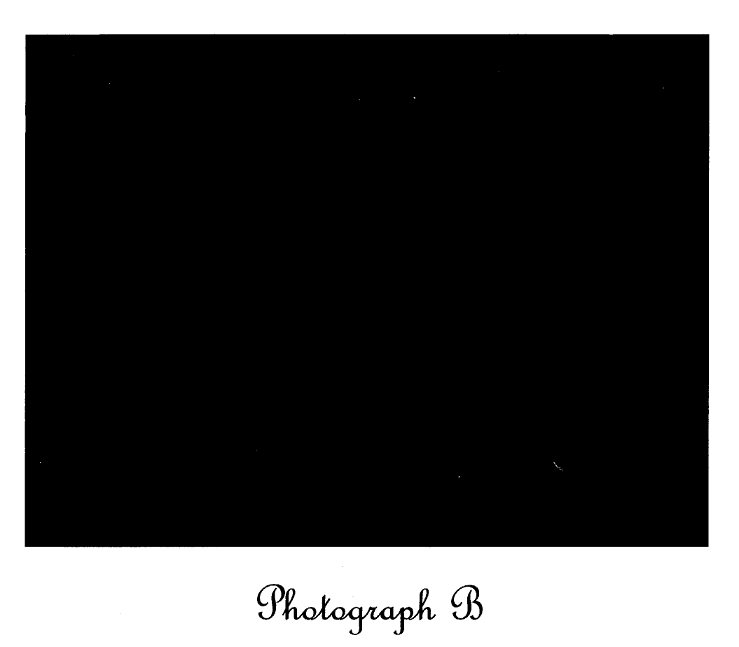

### BACKGROUND OF THE INVENTION

This invention relates to a new and distinct cultivar of Coleus plant, botanically known as Coleus×hybridus, and hereinafter referred to by the cultivar name ‘Painted Pink’. This new coleus is marketed under the trade name “Painted Pink” for nursery, landscape and other ornamental uses typical of coleus.

The new Coleus was discovered by the Inventor in Indian Trail, N.C. on a naturally-occurring lateral branch mutation on a plant of unpatented Coleus×hybridus cultivar ‘Maroon’ in 2002.

The new Coleus has shown that its unique features and characteristics are stable and reproduce true to type in successive generations through asexual reproduction by terminal and stem cuttings taken at Indian Trail, N.C.

### SUMMARY OF THE INVENTION

The following traits have been repeatedly observed and are determined to be the unique characteristics of ‘Painted Pink’. These characteristics in combination distinguish ‘Painted Pink’ as a new and distinct Coleus cultivar:

1. Mounding, outwardly spreading, compact plant habit.

2. Freely branching plant habit.

3. Lack of flower development.

4. Hot pink and maroon bi-color leaves with maroon in the center surrounded by a hot pink margin which size and shape depend on light levels

5. Tolerant to high temperatures and full sun.

Plants of the new Coleus can be compared to the plants of the parent cultivar Coleus×hybridus cultivar ‘Maroon’. Leaves of the ‘Painted Pink’ are bi-color hot pink and maroon in color, whereas the leaves of the parent plant are solid dark maroon in color. Also, in side-by-side comparisons conducted in Indian Trail, N.C. ‘Painted Pink’ were more compact than the parent plant ‘Maroon’.

### DESCRIPTION OF THE PHOTOGRAPHS

The accompanying colored photograph A illustrates the overall appearance ‘Painted Pink’, showing colors as true as possible in colored reproduction of this type. This photograph exhibits a diagonal view of a typical 25 cm hanging basket container of the new cultivar with three plants per container at about 12 weeks after planting of rooted cuttings.

Photograph B illustrates a close-up photo of the texture of ‘Painted Pink’. This photograph shows colors as true as possible in colored reproduction of this type of a typical 25 cm hanging basket container of the new cultivar with three plants per container at about 12 weeks after planting of rooted cuttings.

### BOTANICAL DESCRIPTION

The cultivar ‘Painted Pink’ has not been observed under all possible environmental conditions. The Phenotype may vary somewhat with variations in environmental conditions such as light intensity, temperature, light duration, and fertility levels without however any variance in genotype.

In the following description, color references are made to The Royal Horticultural Society Colour Chart, except where general terms of ordinary dictionary significance are used.

Parentage: Naturally-occurring branch mutation of Coleus×hybridus cultivar ‘Maroon’.

Propagation:

Type cutting.—Terminal stem cuttings.

Time to initiate roots.—Summer: About 5 days at 30 degree C. Winter: About 6 days at 21 degree C.

Time to develop roots.—Summer: About 21 days at 30 degree C. Winter: About 28 days at 21 degree C.

Rooting habit.—Numerous and freely branching.

Plant description:

Botanical classification: Coleus×hybridus cultivar ‘Painted Pink”.

General appearance.—Mounding, outwardly spreading, compact plant habit.

Crop time.—From unrooted cuttings about 10 weeks are required to produce a fully-grown finished plant in a 10 cm container.

Vigor.—moderate.

Lateral branches.—Freely branching; lateral branches may potentially form at every node.

Plant height.—About 20 cm from soil level to the top of the plant.

Plant diameter.—About 13 cm.

Branch description.—Diameter about 4 cm. Internode length about 2 cm. Shape: square Color: 187B.

Foliage description.—Arrangement: Opposite. Division: Simple. Length: About 7.2 cm. Width: About 5 cm. Shape: Ovate. Apex: Acuminate. Base: Aequilateral. Margin: Crenate: Texture: upper surface: Coarse, Strumose. lower surface: Coarse, Cacellate Venation pattern: Net-veined. Color: Young foliage, lower surface center, maroon, 187A. Young foliage, lower surface outer, hot pink, 70A. Young foliage, upper surface center, maroon, 187A. Young foliage, upper surface outer, hot pink, 71C. Fully expanded foliage, lower surface center, maroon, 187A. Fully expanded foliage, lower surface outer, hot pink 72A. Fully expanded foliage, upper surface center, maroon, 187A. Fully expanded foliage, upper surface outer, hot pink, 71B. Center is maroon in color surrounded by hot pink outer area. Actual size and shape of outer area is dependent on light levels. Higher light levels increase the area of the hot pink color.

Petiole: Length about 3.6 cm. Diameter about 2.1 mm. Color 187A.

Flower description: Flowers have not been observed to date on plants of ‘Painted Pink’.

I claim:

1. A new and distinct cultivar of Coleus plant named ‘Painted Pink’, as illustrated and described.
